(19) United States
(12) Patent Application Publication
(10) Pub. No.: US 2020/0129546 A1
(43) Pub. Date: Apr. 30, 2020

(54) TREATMENT OF BLEPHARITIS

(71) Applicant: FINE COTTON FACTORY INC., Toronto (CA)

(72) Inventor: Biren PATEL, Toronto (CA)

(73) Assignee: FINE COTTON FACTORY INC., Toronto, ON (CA)

(21) Appl. No.: 16/624,625

(22) PCT Filed: Jun. 19, 2018
(86) PCT No.: PCT/CA2018/050743

§ 371 Date: Dec. 19, 2019

Related U.S. Application Data
(60) Provisional application No. 62/521,832, filed on Jun. 19, 2017.

(51) Int. Cl. A61K 33/34; A61K 9/00; A61K 9/70; A61K 9/12; A61K 47/38; A61P 27/02; A61P 31/04; A61P 17/00

(52) U.S. Cl. CPC A61K 33/34; A61K 9/0048; A61K 9/7007; A61P 17/00; A61K 47/38; A61P 27/02; A61P 31/04; A61K 9/12

(57) ABSTRACT

Copper-containing articles and methods for reducing, preventing or treating blepharitis, red eye, comprising having a subject in need thereof in proximity to or in contact with an article containing copper.

### FIELD OF THE INVENTION

The present invention relates to a device containing copper useful for reducing the symptoms, treatment or prevention of blepharitis.

### BACKGROUND OF THE INVENTION

Blepharitis is a common eye condition characterized by chronic inflammation of the oil glands that are located behind the eyelashes, resulting in inflamed, irritated, itchy, and reddened eyelids. A number of diseases and conditions can lead to blepharitis. It can be caused by the oil glands at the base of the eyelashes becoming clogged, a bacterial infection, allergies, or other conditions. The severity and course can vary. Onset can be acute, resolving without treatment within 2-4 weeks (this can be greatly reduced with lid hygiene), but more generally is a long-standing chronic inflammation of varying severity, and is difficult to treat.

Blepharitis may be associated with skin conditions such as ocular rosacea, seborrheic dermatitis and psoriasis. Often, blepharitis and pink eye occur at the same time.

Blepharitis symptoms and signs include: watery eyes, red eyes, gritty, burning or stinging sensation in the eyes, eyelids that appear greasy, itchy eyelids, red, swollen eyelids, flaking of the skin around the eyes, crusted eyelashes upon awakening, eyelid sticking, more frequent blinking, sensitivity to light, eyelashes that grow abnormally (misdirected eyelashes), loss of eyelashes. A person having blepharitis may also experience eyelash problems, eyelid skin problems, excess tearing or dry eyes, difficulty wearing contact lenses, sty, chalazion, chronic pink eye, injury to the cornea.

Staphylococcal blepharitis is caused by infection of the anterior portion of the eyelid by Staphylococcal bacteria. Symptoms include a foreign body sensation, matting of the lashes, and burning. Collarette around eyelashes, a ring-like formation around the lash shaft, can be observed. Other symptoms include loss of eyelashes or broken eyelashes. The condition can sometimes lead to a chalazion or a stye.

Staphylococcal blepharitis is a condition which may start in childhood and continue through adulthood. It is commonly recurrent and it requires special medical care. The prevalence of Staphylococcus aureus in the conjunctival sac and on the lid margin varies among countries, probably due to climate. Chronic bacterial blepharitis may lead to ectropion.

Posterior blepharitis or rosacea-associated blepharitis is inflammation of the eyelids secondary to dysfunction of the meibomian glands. Like anterior blepharitis, it is a bilateral chronic condition and is manifested by a broad spectrum of symptoms involving the lids including inflammation and plugging of the meibomian orifices and production of abnormal secretion upon pressure over the glands. It may be associated with skin rosacea, and there is growing evidence that in some cases it is caused by demodex mites.

Blepharitis does not often disappear entirely, and even successful treatment is often followed by relapses.

A Cochrane Systematic Review of topical antibiotics were shown to be effective in providing symptomatic relief and clearing bacteria for individuals with anterior blepharitis. Topical steroids provided some symptomatic relief but were ineffective in clearing bacteria from the eyelids. Lid hygiene measures such as warm compresses and lid scrubs were found to be effective in providing symptomatic relief for participants with anterior and posterior blepharitis.

Clearly, in view of the foregoing, it would be advantageous to provide for a treatment that does not include the use of drug-based antibiotics and that would prevent relapses.

There is a need for articles or devices and methods for reducing or treating blepharitis, particularly bacterial-induced blepharitis, that does not involve the use of anti-biotic or topical steroids or that can be used in combination with other therapies.

U.S. Pat. No. 9,469,923 (US'923) describes synthetic fibers with copper and suggests that copper containing products can be used to control odor caused by bacteria, or inhibiting bacterial growth on the article/device itself. However, US'923 does not enable any of these uses, nor describe or suggest copper containing copper in the treatment of conditions or diseases, as it only describes anti-odor and hygiene benefits. Furthermore, US'923 does not describe or suggest that copper containing articles can be used in the treatment of conditions or diseases caused by bacteria found under an epidermal layer, or in oil glands.

### SUMMARY OF THE INVENTION

The present invention relates to a device containing copper that aid in reducing the symptoms, treatment or prevention of blepharitis and prevent relapses.

In one embodiment the present invention is a method of reducing symptoms, preventing or treating blepharitis or dry eye, comprising disposing an article or device containing copper in proximity to or in contact with an affected eyelid of the subject.

In one embodiment of the method of reducing symptoms, preventing or treating blepharitis, dry eye, the article or device containing copper is a fabric or a foam.

In another embodiment of the method of reducing, preventing or treating blepharitis, dry eye, the concentration of copper in the article or device by weight is between about 0.01% to 99.9%.

In another embodiment of the method of reducing, preventing or treating blepharitis, dry eye, the copper is in metallic form, salts or ionic form, wherein the metallic form includes particulates, alloys and oxides.

In another embodiment of the method of reducing, preventing or treating blepharitis, dry eye, the article or device is a fabric, the fabric being made of a yarn comprising copper and a polymer or cellulose or regenerated cellulose.

In another embodiment of the method of reducing, preventing or treating blepharitis, dry eye, the concentration of copper in the yarn is between 0.01% and 99.9%.

In another embodiment of the method of reducing, preventing or treating blepharitis, dry eye, the concentration of the yarn in the fabric is between 0.01% and 100%.

In another embodiment, the present invention relates to a fabric having anti-microbial infection, anti-blepharitis, and/or anti-dry eye properties, the fabric comprising an effective amount of copper.

In one embodiment of the fabric having anti-microbial infection, anti-blepharitis, and/or anti-dry eye properties, the copper concentration in the fabric by weight is between about 0.01% to 99.9%.

In another embodiment of the fabric having anti-microbial infection, anti-blepharitis, and/or anti-dry eye properties, the copper is in metallic form, salts or ionic form, wherein the metallic form includes particulates, alloys and oxides.

In another embodiment of the fabric having anti-microbial infection, anti-blepharitis, and/or anti-dry eye properties, the fabric is made of a yarn comprising copper and a polymer or cellulose or regenerated cellulose.

In another embodiment, the present invention relates to the use of an article or device containing copper for reducing, preventing or treating microbial infection, blepharitis and/or dry eye.

In one embodiment of the use the article or device containing copper, the article or device containing copper is a fabric or a foam.

In another embodiment of the use the article or device containing copper, the copper concentration in the article by weight is between about 0.01% to 99.9%.

In another embodiment of the use the article or device containing copper, the copper is in metallic form, salts or ionic form, wherein the metallic form includes particulates, alloys and oxides.

In another embodiment, the present invention provides for a fabric comprising an effective amount of copper for use in the manufacture of an article having anti-microbial infection, anti-blepharitis, and anti-dry eye properties.

In one embodiment of the fabric of the present invention, the copper concentration in the fabric by weight is between about 0.01% to 99.9%.

In another embodiment of the fabric of the present invention, the copper is in metallic form, salts or ionic form, wherein the metallic form includes particulates, alloys and oxides.

In another embodiment of the fabric of the present invention, the fabric is made using a yarn comprising copper and a polymer or cellulose or regenerated cellulose.

In another aspect according to any of the previous embodiments, the article or devices include (i) top of the bed product, (ii) mattress and mattress covers, (iii) removable mattress toppers, (iv) pillow cases, (v) pillow shell, (vi) pillow fill, (vii) blanket and blanket fill, (viii) fitted and loose sheets for bedding, (xi) mattress pads, (xii) chair liners, (xiii) sleeping bags, (xiv) clothing, including pajamas, socks and so forth, (xv) towels, (xvi) in hospital settings in mattress/toppers/removable covers/all related to mattress or any resting or sleeping surface, (xvii) in mattresses, (xviii) a sleep mask, (xix) hat, or (xx) glasses.

### BRIEF DESCRIPTION OF THE DRAWINGS

The following figures illustrate various aspects and preferred and alternative embodiments of the invention.

FIG. 1: illustration of a knitting sequence of a copper-containing fabric according to one embodiment of the present invention.

FIG. 2: illustration of a knitting sequence of a copper-containing fabric according to another embodiment of the present invention.

FIG. 3: illustration of a knitting sequence of a copper-containing fabric according to another embodiment of the present invention.

FIG. 4: illustration of a knitting sequence of a copper-containing fabric according to another embodiment of the present invention.

### DESCRIPTION OF THE INVENTION

### Definitions

The following definitions, unless otherwise stated, apply to all aspects and embodiments of the present application. Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “include without limitation”). Singular forms included in the claims such as “a”, “an” and “the” include the plural reference unless expressly stated otherwise. All relevant reference, including patents, patent applications, government publications, government regulations, and academic literature are hereinafter detailed and incorporated by reference in their entireties.

The terms “population” or “plurality,” when used herein, is defined as two, or more than two. The term “another,” as used herein, is defined as at least a second or more. The phrases “at least one of . . . and . . . ” or “one or more” when used herein refer to and encompasses any and all possible combinations of one or more of the associated listed items. As an example, the phrase “at least one of A, B and C” includes A only, B only, C only, or any combination thereof (e.g. AB, AC, BC or ABC). The term “substantially” includes exactly the term it modifies and slight variations therefrom.

The term “about” modifying any amount refers to the variation in that amount encountered in real world conditions of producing materials such as polymers or composite materials, e.g., in the lab, pilot plant, or production facility. For example, an amount of an ingredient employed in a mixture when modified by about includes the variation and degree of care typically employed in measuring in a plant or lab and the variation inherent in the analytical method. Whether or not modified by about, the amounts include equivalents to those amounts. Any quantity stated herein and modified by “about” can also be employed in the present invention as the amount not modified by about. In order to aid in the understanding and preparation of the within invention, the following illustrative, non-limiting, examples are provided.

The term “knit” may be used as a verb (to make (a fabric or garment) by interweaving yarn or thread in a series of connected loops either by hand, with knitting needles, or on a machine) or as a noun (a fabric or garment made by knitting).

“Fiber” and “fibers” refer to any slender, elongated structure that can be carded or otherwise formed into a thread. Examples of fibers include “stable fibers,” a term that is well-known in the textile art.

“Subject” refers to any mammal, including both humans and non-human mammals.

“Yarn” refers to a structure comprising a plurality of threads.

“Dispose” as used herein is meant placing or arranging the copper containing article or device of the present invention in proximity to or in contact with a body part of interest, for example in the case of blephartis, in proximity to or in contact with an affected eyelid.

“Effective amount” as used herein is meant an amount of copper content in an article or device, high enough to significantly positively modify the condition to be treated but low enough to avoid serious side effects (at a reasonable benefit/risk ratio), within the scope of sound medical judgment.

The present invention discloses the use of copper-containing articles or devices in methods for the treatment of blepharits and red eye. The therapeutic properties of the copper-containing articles of present invention have effect even when the bacteria is located under the epidermal layer or in an oil gland. Furthermore, the inventors also discovered that the copper-containing articles of the present invention can be used in the treatment of nail fungus. The applicant has demonstrated that nail fungus was healed in a week after having the nail fungus in close proximity with a copper-containing article of the present invention.

The copper-containing articles or devices of the present invention can be used to treat and reduce the symptoms of blepharitis and red eyes. The applicant has shown that copper contained in articles such a sleeping mask reduced the symptoms of blepharitis and red eyes within one day of use. That articles containing copper can reduce and treat the symptoms of blepharitis is surprising. Blepharitis is typically caused by bacterial infection or blockage of the eyelid's oil glands. That is, the bacteria are not exposed and do not come into direct contact with the copper contained in the articles of the present invention.

The textiles or fabrics of the present invention may be manufactured with copper-containing yarns, or may be infused, embedded or impregnated with copper. Foams may be infused, embedded or impregnated with copper. The copper-may be in metallic form (e.g., particulates, alloys and oxides), salts and/or ionic form.

In another embodiment, the copper may be provided on its own. For example, a subject may sleep in the proximity of or in contact with copper or copper containing objects or articles, such as copper coins, cables with copper wires, naked copper wires, and so forth. By “in the proximity” is meant that the subject is not in direct contact with the copper, but effectively or sufficiently close to the copper to positively exert its anti-concussion/acne/neuropathy properties. The proximity may vary with the duration of the exposure, the nature of any barriers between the copper-containing article and the subject, the number of barriers between the copper-containing article and the subject, the distance between the copper and the subject or any combination thereof. For example, the copper may be restricted to the inlay of a three-layer fabric, as such there would be at least one layer of fabric between the copper and the subject. Subject to the nature of the barrier, the effective proximity to exert anti-sleep disorder properties will thus be the maximum distance between the copper in the article and the subject, or the maximum number of barriers between the copper and the subject which will provide the desired effect on the subject's sleep disorder. In another embodiment, the subject may apply to a body surface exhibiting the acne or the neuropathy, or wrap around the head (in the case of concussion) the copper-containing article of the present invention for an effective amount of time.

The copper containing articles of the present invention were found to have a therapeutic or ameliorating effects even at one week of the subject using a mask made with the copper-containing fabric of the present invention. As such, the subject may, for example, sleep in the proximity of or in contact with or apply the copper containing article of the present invention for less than one day, one day, more than one day, one week, more than one week, one month, more than one month, a year or even more than one year. It is understood that the subject does not need to be asleep. For example, the subject may just wear an article of the present invention in proximity to the affected area, i.e. the subject's eye.

The copper-containing articles or devices of the present invention can also be used in methods of inhibiting, reducing or preventing blepharitis or red eye in a subject, by disposing an article of the present invention in close proximity of or in contact with the eye area of a subject.

As such, in one embodiment, the present invention provides for a method of reducing, preventing or treating blepharitis or dry eye in a subject in need, comprising disposing an article or device containing copper in proximity or in contact with an eye of the subject.

The textile or fabric of the present invention may be made using a yarn comprising a polymer or cellulose, or re-generated cellulose, or otherwise, and copper. The copper may be provided as a powder, liquid or alloy forms. Polymers include a compound suitable for fiber and fabric generation including a thermoplastic polymer, polyester, nylon, rayon, polyethylene (PE), polypropylene (PP), polyethylene terephthalate (PET), polyethylene terephthalate glycol (PETG), co-PET, polylactic acid (PLA), and polytrimethylene terephthalate (PTT).

The fabric, in one embodiment, may be formulated in a jersey.

In some embodiment, the yarn of the present invention may be copper-containing fiber. An example of a copper-containing fiber is described in WO/2012/088507, the content of which is incorporated herein by reference.

In another embodiment, the textiles or fabrics of the present invention may contain copper attached, coupled or connected to the fabric, or the copper may be loose. For example, the fabric may be in the form of a bag having copper inside the bag in loose form or attached to the inside walls of the bag, or copper may be attached, connected, or coupled to an existing fabric. Alternatively, the fabric may contain one or more pockets into which the copper can be contained in loose or attached form. The pockets may be designed to be in a closed or opened from. For example, the pocket may include attachment means such as buttons or Velcro® or may include a flap to open or close the pocket. This design of pockets may allow a person to insert or remove the copper from the pockets. Copper may be provided in any form as previously described. For example, the copper may be provided as a powder, liquid, alloys, filaments, wires, or in the form of coins (for example one cent coins known as pennies). The copper may be provided in metallic form, salt from or ionic form.

The fabric of the present invention may have one, two, three, four or more than four layers. A three layer fabric (with inlay), ticking fabric and/or double knit fabric (two layer without inlay), and/or a single knit construction of any kind, or a woven of any kind or content. The copper may be included any one of the layers of the fabric, and in more than one layer. For example, in a two layer fabric, the copper may be included in both layers, or in just one layer. In a three layer fabric having a top, middle and bottom layers, the copper may be included in just one of the 3 layers (in any of the top, middle or bottom layers), in 2 of the 3 layers (in any combination of two of the top, middle or bottom layers) or in all of the 3 layers.

The applicant surprisingly discovered that the benefits of copper for blepharitis and red eyes, can be achieved even when the copper is contained in the inlay of the fabric, i.e. not in direct contact with the skin of the subject using the fabric. Tests on certain fabrics having the copper only in the inlay have been shown not to release copper ions. As such, the results of obtained in Example 1 below, in which the copper was restricted to the inlay (i.e. not in contact with the study participant) are surprising.

In the case of a three layer fabric, yarn position on the three layer fabric may be as follows:

1) All face feeds: copper-containing fiber; all inlay feeds: polymer filaments; and/or all back feeds: polymer filaments.

2) All face feeds: polymer fiber; all inlay feeds: copper-containing fiber; and/or all back feeds: polymer filaments.

3) All face feeds: polymer filaments; all inlay feeds: polymer filaments; and/or all back feeds: copper-containing fiber.

The yarn can be used by itself or with any other known and unknown fibers. Known fibers, such as Celliant, coolmax, cellulose fibers, FR. Fibers, infrared yarn, natural or manmade fiber, either as outer layer or inner layer may be used. The Cu concentration in the yarn can be anywhere between 0.01% to 99.9% by weight (wt.) including 0.05% 1%, 1.5%, 2%, 2.5%, 3%, 3.5%, 4%, 4.5%, 5%, 5.5%, 6%, 6.5%, 7%, 7.5%, 8%, 8.5%, 9%, 10%, 15%, 20%, 25%, 30%, 35%, 40%, 45%, 50%, 55%, 60%, 65%, 70%, 75%, 80%, 85%, 90%, 91.2%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.9% by weight and any percentage by weight there in between. In one embodiment, the Cu device in the yarn is between about 0.05% to 4.5% wt. Total content of this Cu-containing yarn in the fabric or textile of the present invention may be anywhere between about 0.01% and 100% of the total in the fabric, including 0.05%, 1%, 1.5%, 2%, 2.5%, 3%, 3.5%, 4%, 4.5%, 5%, 5.5%, 6%, 6.5%, 7%, 7.5%, 8%, 8.5%, 9%, 10%, 15%, 20%, 25%, 30%, 35%, 40%, 45%, 50%, 55%, 60%, 65%, 70%, 75%, 80%, 85%, 90%, 91.2%, 91.5%, 92%, 92.5%, 93%, 93.5%, 94%, 94.5%, 95%, 95.5%, 96%, 96.5%, 97%, 97.5%, 98%, 98.5%, 99%, 99.5%, 100% and any percentage there in between.

This yarn may be spun or filament and may be used on one layer or in multiple layer fabrics. The fabric can be made using known technologies, such as knitting technology, woven technology or non-woven technology.

The fabric may be made using elastane yarn (i.e. Spandex® or Lycra®).

The copper-containing fabric or textile of the present invention that can be used to treat microbial infections, inflammation, blepharitis, dry eye and so forth may be manufactured by methods known in the art. In one embodiment, the process for making the copper-containing fabric properties includes:

(a) knitting a fabric on a circular weft knitting machine (or dial selection machine, or any other such type of knitting or weaving machine as may be invented during the term of this patent) using a group of yarns consisting of various fibers or a combination thereof;

(b) introducing a copper thread or yarn to the back of the fabric, referring to the dial in any combination of other fibers; or

(c) introducing a copper thread or yarn to the face of the fabric, referring to the cylinder in any combination of other fibers; or

(d) introducing a copper thread as a filler, referring to the space between the face and the back of the fabric in any combination of other fibers.

The knitting machine may be of various diameters and gauges and a combination thereof. The thread or yarns may be of various colors, counts or deniers and any combination thereof.

The process of manufacture may also include the use of various knitted structures and a combination thereof. Full and half gauge structures are also included and a combination thereof.

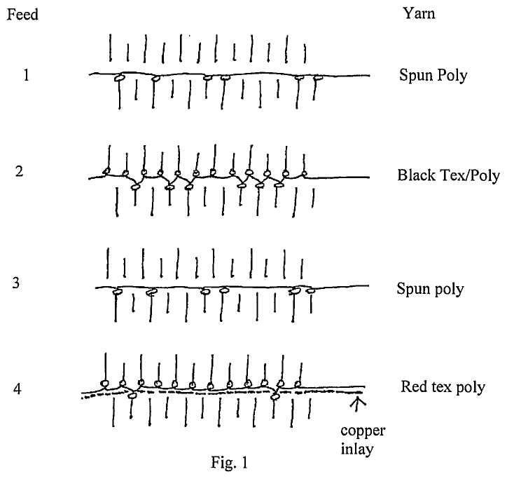

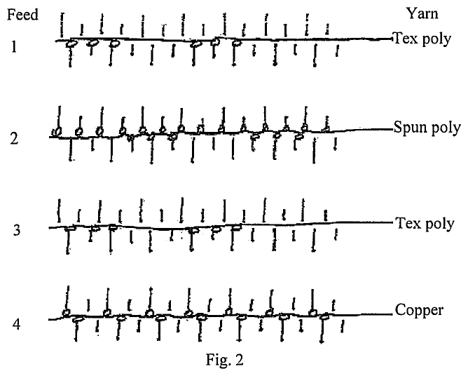

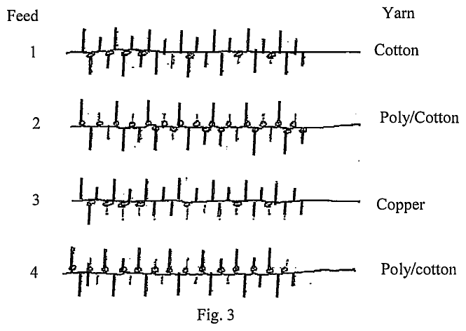

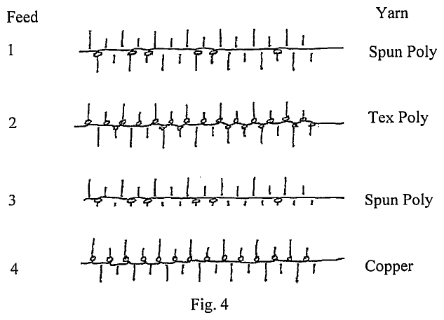

FIGS. 1-4 illustrate non-limiting knitting sequences of a copper-containing fabric according to different embodiments of the present invention.

Tables 1-4 illustrate the knitting sequences for FIGS. 1-4 respectively.

TABLE 1

| Feed # | Dial set out | Cylinder set out | Yarn |
|---|---|---|---|
| 1 | Welt all | Select as per design | Spun poly |
| 2 | Knit all | Select as per design | Black tex/Poly |
| 3 | Welt all | Select as per design | Spun Poly |
| 4 | Welt all | Select as per design | Red Tex/Poly + |
| | | | copper inlay* |

*does not knit, the copper is sandwiched between face and back of fabric

TABLE 2

| Feed # | Dial set out | Cylinder set out | Yarn |
|---|---|---|---|
| 1 | Welt all | Select as per design | Tex poly |
| 2 | Knit all | Select as per design | Spun Poly |
| 3 | Welt all | Select as per design | Tex Poly |
| 4 | Knit all | Select as per design | Copper** |

**Copper is knitting on dial and cylinder feed and showing on both face and back of fabric.

TABLE 3

| Feed # | Dial set out | Cylinder set out | Yarn |
|---|---|---|---|
| 1 | Welt all | Select as per design | Cotton |
| 2 | Knit all | Select as per design | Poly/cotton |
| 3 | Welt all | Select as per design | Copper*** |
| 4 | Knit all | Select as per design | Poly/cotton |

***Copper is knitting on cylinder only feed and showing on face only.

TABLE 4

| Feed # | Dial set out | Cylinder set out | Yarn |
|---|---|---|---|
| 1 | Welt all | Select as per design | Spun poly |
| 2 | Knit all | Select as per design | Tex/Poly |
| 3 | Welt all | Select as per design | Spun Poly |
| 4 | Knit all | Select as per design | Copper**** |

****Copper is knitting on dial only feed and showing on back only.

Alternatively, the fabric containing copper of the present invention may include a woven or non-woven textile that has been impregnated in or coated with copper. Jeffrey Gabbay et al. (J. of Industrial Textiles, vol. 35, issue 4, 2006, p. 323, the contents of which are incorporated herein by reference) provide an example on impregnating textiles with copper oxide. “Briefly, cotton fibers are plated as follows: cationic copper (a mix of Cu²⁺ and Cu¹⁺) is bound to cellulose-based fibers. Cotton, rayon/viscose, and fibers such as lyocel by Tencel are treated with an electroless plating process, which includes the following steps: (a) fibers having a diameter of about 11-13 mm are soaked for 5 s in 1% SnCl2, pH 3.5 at room temperature; (b) the fibers are then soaked for 5 s in PdCl2, pH 4 at room temperature, producing activated fibers; and (c) the activated cellulose based fibers are then exposed to formaldehyde, CuSO₄, and polyethylene glycol at pH 9. After about 5 min the fibers are plated with cationic copper (Cu(II) and Cu(I)). Finally, the fibers are dried and run through a textile carding machine which separates and aligns them.

“Impregnation of copper into the various synthetic fibers mentioned above is achieved by adding a cupric oxide powder to the polymers during the master batch preparation stage. The master batch can be made in industrially accepted concentrations and added to the polymeric slurry the same way any other master batch would be added, such as for pigmentation, etc. The copper oxide doped master batch is designed in such a way as to allow fiber extrusion in the normal production systems. The fibers can be cut into short staple or produced in filament form and texturized, if so desired. As in the case of the plated fibers mentioned above, the product yielded is a fiber that can be introduced at the blending stage of yarn production or directly into a woven or knit product so that no manufacturing processes are changed.” (See Jefferey Gabbay et al (supra) at p. 325).

In this document, “article” means a particular item or object capable of being placed in proximity to or in contact with an eyelid area of a subject. The articles or devices of the present invention, including foam, fabric or textile may have different applications, including (i) top of the bed product, (ii) mattress and mattress covers, (iii) removable mattress toppers, (iv) pillow cases, (v) pillow shell, (vi) pillow fill, (vii) blanket and blanket fill, (viii) fitted and loose sheets for bedding, (xi) mattress pads, (xii) chair liners, (xiii) sleeping bags, (xiv) clothing, including pajamas, socks and so forth, (xv) towels, (xvi) in hospital settings in mattress/toppers/removable covers/all related to mattress or any resting or sleeping surface, (xvii) in mattresses, (xviii) a sleep mask, (xix) hat, or (xx) eye glasses.

In order to aid in the understanding and preparation of the within invention, the following illustrative, non-limiting, examples are provided.

### Example 1

A female participant suffering of blepharitis and dry eye was provided with a sleeping mask made with copper containing fabric.

The participant has suffered blepharitis for several years. However, she was not responding to the traditional treatments.

A sleeping mask made of copper-containing fabric of the present invention (34% copper spun on the face, 39% copper inlay made as a 3 layer ticking) was provided to the participant and instructed to sleep with it. After one week of sleeping with the mask, the participant was asked to fill the following questionnaire:

You are receiving this survey because you have received a test sample. In providing you with this sample, our expectation is that you would contribute to the research pool by sharing your experience in using the product no matter what the result is. Once the survey is complete, please return it to the contact information specified below. Again, thank you for your interest.

1. What are your current discomforts or symptoms are you experiencing? Answer: very dry eyes, blepharitis.

2. How long has this been going on?

Answer: several years.

3. Are you currently working with a doctor? If not please explain.

Answer: Yes I am.

4. Do you have test results that are used to evaluate your current issues? Answer: There aren't test results for this—it is diagnosed by the eye doctor.

The participant filled the following table as follows:

Please list each of your symptoms below
Rate the improvement for each of the symptoms on a scale of 1, 2, or 3 below
1—there is no improvement
2—you have received some improvement
3—you no longer have the symptom

| Symptom | Before use | 1 week | 2 weeks | 3 weeks | 4 weeks |
|---|---|---|---|---|---|
| Dry Eyes | 1 | 2 | 2 | 2 | 3 |
| Blepharitis | 1 | 2 | 2 | 2 | 3 |

This participant has stopped using eye drops and no longer has symptoms of blepharitis.

### Example 2

A female participant suffering of blepharitis, rosacea (a chronic inflammatory skin condition) was provided with a sleeping mask made with copper containing fabric.

A sleeping mask made of copper-containing fabric of the present invention (34% copper spun on the face, 39% copper inlay made as a 3 layer ticking) was provided to the participant and instructed to sleep with it. After four weeks of sleeping with the mask, the participant was asked to fill the following questionnaire.

You are receiving this survey because you have received a test sample. In providing you with this sample, our expectation is that you would contribute to the research pool by sharing your experience in using the product no matter what the result is. Once the survey is complete, please return it to the contact information specified below. Again, thank you for your interest.
1. What are your current discomforts or symptoms are you experiencing? ______
   a. I used to have blepharitis before I started using the copper bedsheet and pillowcase. I now no longer have blepharitis, an eye condition.
   b. I had a lesion on my lower eyelid that my doctor told me would have to be removed—he said it would not get better on its own.
   c. I have rosacea, and it is significantly better since I started using the copper-infused pillowcase.
2. How long has this been going on? ______
   a. My blepharitis was going on for a few years.
   b. my lesion was in place for a few months
   c. My rosacea was going on for years.
2. Are you currently working with a doctor? If not please explain.
   a. Yes, with an eye surgeon and an opthamologist.
   b. with my opthamologist
   c. Rosacea—my GP.
3. Do you have test results that are used to evaluate your current issues? ______
   a. At my last checkup in the Fall, 2017, my ophthalmologist told me that I no longer have blepharitis.
   b. My lesion is fully gone
   c. No test results re Rosacea, but the redness is significantly better, and the bumps have lessened. ______
      The following questions are so we can collect information on any of the benefits you have received during your experience with the product.
Please list each of your symptoms below
Rate the improvement for each of the symptoms on a scale of 1, 2, or 3 below
   1—there is no improvement
   2—you have received some improvement
   3—you no longer have the symptom

| | | | | | |
|---|---|---|---|---|---|
| Symptom | Before use | 1 week | 2 weeks | 3 weeks | 4 weeks |

| | | | | |
|---|---|---|---|---|
| Blepharitis - eyes | 2 | 2 | 2 | 3 |
| Rosacea on face | 1 | 1 | 2 | 2 |
| Eyelid Lesion | 1 | 1 | 1 | 3 |

As it can be seen, this participant no longer had blepharitis.

### Example 3

A female participant suffering of Blepharitis, rosacea (a chronic inflammatory skin condition) was provided with a sleeping mask made with copper containing fabric.

A sleeping mask made of copper-containing fabric of the present invention (34% copper spun on the face, 39% copper inlay made as a 3 layer ticking) was provided to the participant and instructed to sleep with it. After four weeks of sleeping with the mask, the participant was asked to fill a questionnaire.
1. What are your current discomforts or symptoms are you experiencing? Answer: Oily eyelids, heaviness in eyes, on waking blurry Vision and red irritated eyes, crusty and dozing eyes.
2. How long has this been going on?
   Answer: About 6 months
3. Are you currently working with a doctor? If not please explain.
   Answer: Not working with a doctor as there doesn't seem much they can do that we can't do on our own.
4. Do you have test results that are used to evaluate your current issues?
   Answer: No
5. Can you identify the exact strain of the condition that we are sending you the test kit for?
   Answer: Blepharitis.
      The following questions are so we can collect information on any of the benefits you have received during your experience with the product.

Please list each of your symptoms below
Rate the improvement for each of the symptoms on a scale of 1, 2, or 3 below
   1—there is no improvement
   2—you have received some improvement
   3—you no longer have the symptom

| Symptom | Before use | 1 week | 2 weeks | 3 weeks | 4 weeks |
|---|---|---|---|---|---|
| Oily lids | 1 | 2 | 2 | 3 | 3 |
| Itchy eyes | 2 | 2 | 3 | 3 | 3 |
| Puffy eyes | 1 | 2 | 3 | 3 | 3 |
| Red eyes | 1 | 1 | 2 | 2 | 3 |
| Dozing Eyes | 1 | 2 | 2 | 3 | 3 |
| Blurry vision | 1 | 1 | 1 | 2 | 3 |
| Heaviness in eyes | 1 | 2 | 2 | 3 | 3 |
| Crusty eyes | 2 | 2 | 3 | 3 | 3 |

As it can be seen, this participant no longer has symptoms of blepharitis, i.e. oily lids, itchy eyes, puffy eyes, red eyes, dozing eyes, blurry vision, heaviness in eyes, an crusty eyes.

Through the embodiments that are illustrated and described, the currently contemplated best mode of making and using the invention is described. Without further elaboration, it is believed that one of ordinary skill in the art can, based on the description presented herein, utilize the present invention to the full extent. All publications cited are incorporated by reference.

1. A method of reducing the symptoms, preventing or treating blepharitis, dry eye or rosacea in a subject in need, comprising disposing a device containing copper in proximity to or in contact with an affected eyelid of the subject.

2. The method of claim 1, wherein the device containing copper is a fabric or a foam.

3. The method of claim 1, wherein the concentration of copper in the device by weight is between about 0.01% to 99.9%.

4. The method of claim 1, wherein the copper is in metallic form, salts or ionic form, and wherein the metallic form includes particulates, alloys and oxides.

5. The method of claim 1, wherein the device is a fabric, the fabric being made of a yarn comprising copper and a polymer or cellulose or regenerated cellulose.

6. The method of claim 5, wherein the concentration of copper in the yarn is between 0.01% and 99.9%.

7. The method of claim 5, wherein the concentration of the yarn in the fabric is between 0.01% and 100%.

8. An anti-blepharitic fabric, wherein the fabric comprises an effective amount of copper.

9. The anti-blepharitic fabric of claim 8, wherein copper concentration in the fabric by weight is between about 0.01% to 99.9%.

10. The anti-blepharitis fabric of claim 8, wherein the copper is in metallic form, salts or ionic form, wherein the metallic form includes particulates, alloys and oxides.

11. The anti-blepharitis fabric of claim 8, wherein the fabric is made of a yarn comprising copper and a polymer or cellulose or regenerated cellulose.

12-15. (canceled)

16. A fabric comprising an effective amount of copper for use in the manufacture of a device having anti-blepharitis, anti-dry eye, and anti-rosacea properties.

17. The fabric of claim 16, wherein copper concentration in the fabric by weight is between about 0.01% to 99.9%.

18. The fabric of claim 16, wherein the copper is in metallic form, salts or ionic form, wherein the metallic form includes particulates, alloys and oxides.

19. The fabric of claim 18, wherein the fabric is made using a yarn comprising copper and a polymer or cellulose or regenerated cellulose.

20. The method of claim 2, wherein the concentration of copper in the device by weight is between about 0.01% to 99.9%.

21. The method of claim 2, wherein the copper is in metallic form, salts or ionic form, wherein the metallic form includes particulates, alloys and oxides.

22. The method of claim 3, wherein the copper is in metallic form, salts or ionic form, wherein the metallic form includes particulates, alloys and oxides.

23. The method of claim 4, wherein the device is a fabric, the fabric being made of a yarn comprising copper and a polymer or cellulose or regenerated cellulose.
